(19) United States
(12) Patent Application Publication
(10) Pub. No.: US 2019/0128792 A1
(43) Pub. Date: May 2, 2019

(54) HORIZONTAL SOIL PERMEABILITY TESTING DEVICE

(71) Applicant: Lafayette College, Easton, PA (US)

(72) Inventors: Mary J. S. Roth, Easton, PA (US); Laurie F. Caslake, Easton, PA (US)

(21) Appl. No.: 16/175,346

(22) Filed: Oct. 30, 2018

Related U.S. Application Data
(60) Provisional application No. 62/580,170, filed on Nov. 1, 2017.

(51) Int. Cl. G01N 15/08; G01N 33/24

(52) U.S. Cl. CPC G01N 15/0806; G01N 33/24; G01N 15/082

(57) ABSTRACT

A horizontal permeability device having an inflow tank and an outflow tank, the inflow tank being taller than the outflow tank. Between the inflow tank and outflow tank is a horizontally defined sample cylinder that is connected to openings in each of the inflow and outflow tanks. The outflow tank comprising an outflow valve positioned above the sample cylinder, thereby allowing water to drain from the inflow tank to the outflow tank, while passing through the sample cylinder.

### CROSS REFERENCE TO RELATED APPLICATIONS

This application claims the benefit of U.S. Provisional Application No. 62/580,170, filed Nov. 1, 2017.

### GOVERNMENT SUPPORT

This invention was made with government support under Grant No. CMMI-1632963 awarded by the National Science Foundation. The government has certain rights in the invention.

### FIELD OF INVENTION

This invention is generally related to an apparatus and methods of using the same, and more particularly to a characterization apparatus for testing of soil conductivity (permeability) in saturated soil, and methods of employing the apparatus which advantageously comprises a horizontal chamber for determining permeability of the soil contained in the horizontal chamber.

### BACKGROUND OF THE INVENTION

The permeability of soil is defined as the soil's conductivity to fluid flow. The permeability of soil to fluid flow depends upon the size of voids in the soil, the soil mineral types, and the degree of saturation of the soil (the magnitude of the voids that contain fluid). The rate of fluid flow through saturated soil (in which the voids are full of fluid) is a function of the soil's permeability and the magnitude of the differential in fluid pressure. Pressure gradients exist due to natural effects such as hydraulic gradients (in the case of groundwater) and due to unnatural (forced) gradients. Unnatural (forced) gradients can be imposed by pumping fluid out of the soil or injecting fluid into the soil.

The permeability of different types of soils can vary by orders of magnitude. Knowledge of the permeability of the soil is required to design structures to reduce natural groundwater flow, to estimate flow into a tunnel or through a dam, and to model the subsurface flow of liquid contaminants, among other applications. Similarly, knowledge of the permeability of soils placed as fill materials (such as those used to fill an excavation or natural depression) is required to estimated flow of fluid through those materials.

Soil permeability has historically been measured either using small samples in a laboratory or using field tests. Laboratory measurements of the permeability of natural soils are an imperfect method due to the difficulty of obtaining truly undisturbed soil samples. Special sampling methods can be used to obtain undisturbed cores for testing, but these methods require specific tools to obtain these samples. However, it is oftentimes beneficial to obtain soil permeability measurements for proposed fill materials in a laboratory setting.

Once samples for permeability tests are generated, it is necessary to introduce fluids of interest through these samples. The presence of contaminants in a fluid of interest can impact permeability measurements in some soils and laboratory tests are necessary to determine the impact of the contaminants on the soil permeability. Indeed, the information gained in laboratory tests is invaluable as the flow of fluid and the travel of contaminant plumes in subsurface soils are capable of being mathematically modeled if the soil's permeability is known. However, a problem occurs in laboratory permeability testing when biological processes occur within the fluid, either as part of the testing design or due to contamination in the system.

Adding bacteria to water to encourage certain biological processes that can modify soil properties is a relatively new area of research and study. Specifically, the introduction of biofilm-forming bacteria into soil as a means to reduce soil permeability in a controlled manner and in controlled volumes of soil (the biofilms that are formed fill the voids in the soil making it more difficult for fluid to pass through the soil) is currently being studied and initial results are promising.

Existing laboratory testing devices for measuring hydraulic conductivity use a vertical chamber to test flow of fluids through a sample and these devices involve tubing to bring the fluid to and from the sample. Tubing creates environments that can support the growth of bacteria and biofilms and these growths can create clogging in the tubing. Flow disruptions caused by the clogging can change the outcome of the tests, without the ability to control for these changes. To overcome this issue in tests where bacterial processes are not desired in the soil, sterile, deionized, or RO-DI water is used. However, such fluid is not a representative sample of the fluids flowing through the soil, for example, when leachate from landfill is being tested, or wherein it is desirable to introduce fluid that intentionally encourages the growth of bacteria in the sample.

Herein, an object of the invention is to provide a new apparatus capable of providing small scale permeability testing, which does not rely upon the use of small diameter tubing used in vertical chamber devices.

### SUMMARY OF THE INVENTION

Embodiments of the present disclosure are related to a horizontal permeability testing apparatus and methods of use of the same to calculate soil permeability of a soil sample placed therein.

In a preferred embodiment, an apparatus for testing permeability of a soil sample comprising: an inflow chamber, an outflow chamber, and a sample cylinder, said inflow chamber comprising an opening disposed on one side of the chamber having an opening corresponding to the diameter of the sample cylinder, and the outflow chamber having a corresponding opening of the diameter of the sample cylinder; each of the inflow chamber and outflow chamber comprising attachment means disposed of adjacent to said openings to accept and secure thereto said sample cylinder.

In preferred embodiments, said inflow chamber further comprising a drain valve positioned below the opening disposed of on said inflow chamber, to allow for draining the chamber after a test, and said outflow chamber having both a drain valve similar to that in the inflow chamber and an outflow valve positioned above the opening in the outflow chamber. Preferably, said sample cylinder is open at each end of the cylinder, and is oriented in a horizontal manner between said inflow chamber and said outflow chamber. In further embodiments, said opening at each end of the cylinder comprises a perforated sheet and a screen disposed of adjacent to one another. Preferably, the screen is positioned inside of the perforated sheet, and wherein the perforated sheet engages with the opening in the inflow chamber and the outflow chamber to maintain the sheet and screen in position. Preferably to secure the cylinder to the chambers, the apparatus further comprising a securing protrusion adjacent to the outer edge of each of the openings in the outflow and inflow chambers, and O-rings being disposed of between the securing protrusions and the sample cylinder, which maintain a seal between the sample cylinder and the inflow tank and between the sample cylinder and the outflow tank.

Preferably the soil sample within the apparatus is a fill material or an undisturbed soil sample.

A further embodiment is directed towards a method of determining permeability of a sample of soil comprising: Placing a perforated (support) sheet into a sample cylinder, and placing a screen (filter material) into the cylinder, wherein said perforated sheet and screen are adjacent to one another at one end of said sample cylinder; filling a sample of soil into a sample cylinder; compacting the soil within said sample; placing a second screen on top of said compacted soil, and placing a second perforated sheet adjacent to said second screen; positioning said sample cylinder between an inflow chamber and an outflow chamber, said inflow chamber and outflow chamber comprising openings positioned to accept the opening at each end of said sample cylinder; securing said sample cylinder between said inflow chamber and outflow chamber to be water tight; filling said inflow chamber with a fluid to be tested sufficient to fill the inflow tank and outflow tank to a level to saturate the soil sample; adding additional fluid to said inflow chamber and measure the rate of decrease of the water level in said inflow chamber; and calculating the permeability of said soil sample using the rate of decrease of the water level.

A further preferred embodiment is directed towards an apparatus for testing permeability of a soil sample comprising: an inflow chamber, an outflow chamber, each chamber having a closed base, a side, and an open top, and a sample cylinder, said inflow chamber comprising an aperture disposed on said side of the inflow chamber, and the outflow chamber having a corresponding outflow aperture on said side of the outflow chamber; each of the inflow chamber and outflow chamber comprising attachment means disposed of on an outer face of the side, adjacent to said aperture and outflow aperture, to accept and secure thereto said sample cylinder, wherein said sample cylinder is positioned to allow passage of fluids through the aperture and outflow aperture between the inflow chamber and outflow chamber.

In one embodiment, the apparatus wherein said inflow chamber further comprising a drain valve positioned below the aperture disposed of on said inflow chamber and said outflow chamber having both a drain valve positioned below the outflow aperture and an outflow valve positioned above the outflow aperture in the outflow chamber.

In one embodiment, the apparatus wherein said sample cylinder is open at each end of the cylinder and is oriented in a horizontal manner between said inflow chamber and said outflow chamber. In a further embodiment, the apparatus wherein, wherein said open at each end of the cylinder comprises a perforated sheet and a screen disposed of adjacent to one another. In a further embodiment, the apparatus wherein the screen is positioned inside of the perforated sheet, and wherein the perforated sheet engages against the outer face on the side of the inflow chamber and the outflow chamber to maintain the perforated sheet and screen in position. In a further embodiment, the apparatus wherein the attachment means is an alignment block defining a circumferential perimeter on the outer face of the side and having a diameter larger than the aperture and outflow aperture, and a gasket being disposed of between the alignment block and the sample cylinder, which maintains the sample cylinder between the inflow tank and the outflow tank. In a further embodiment, the apparatus wherein a soil sample is positioned within the sample cylinder. In a further embodiment, the apparatus wherein the gasket is an O-ring. In a further embodiment, the apparatus wherein the perforated sheet and the screen are made of stainless-steel.

A preferred embodiment is directed towards a method of determining permeability of a sample of soil comprising:
a. placing a perforated sheet into a sample cylinder, said sample cylinder having a first and second opening at each end of said cylinder, and placing a screen into the cylinder, wherein said perforated sheet and screen are adjacent to one another at one end of said sample cylinder;
b. filling a sample of soil into a sample cylinder;
c. compacting the soil within said sample;
d. placing a second screen on top of said compacted soil, and placing a second perforated sheet adjacent to said second screen;
e. positioning said sample cylinder between an inflow chamber and an outflow chamber, said inflow chamber and outflow chamber comprising an aperture on said inflow chamber and an outflow aperture on said outflow chamber, said cylinder positioned wherein the first and second openings are engaged to allow passage of fluids through the cylinder between the aperture and outflow aperture;
f. securing said sample cylinder between said inflow chamber and outflow chamber to be water tight;
g. filling said inflow chamber with a fluid to be tested sufficient to fill the inflow tank and outflow tank to a level to saturate the soil sample;
h. adding additional fluid to said inflow chamber and measure the rate of decrease of the water level in said inflow chamber; and
i. calculating the permeability of said soil sample using the rate of decrease of the water level.

In a further preferred embodiment, the method wherein the permeability of said soil is calculated by:

$$k = \frac{Q L}{A t ( {H}_{1} - {H}_{2} )} ln [ \frac{( {H}_{1} - {H}_{0} )}{( {H}_{2} - {H}_{0} )} ] ,$$

wherein L=Length of soil sample, A=Cross-sectional area of soil sample, Q=Volume of fluid that moves through sample during test, t=length of time of test, H1=Height of the fluid sample at start of test, H2=Height of the fluid sample at end of test, and H0=Height of the fluid sample at stasis.

In a further embodiment, the method wherein said fluid comprises bacteria.

A further embodiment is directed towards a horizontal permeability apparatus comprising an inflow tank and an outflow tank, each having a closed base, four sides, each side having an inner and outer face, an open top, and a height, and an outflow tank, comprising an outflow base, four outflow sides, each side having an inner and outer face, an open top and having a height less than the inflow tank; each inflow and outflow tank comprising an aperture disposed on a side on the inflow tank and an outflow aperture on the outflow tank, said aperture and outflow aperture having attachment components disposed of adjacent to and around said aperture on the outer face of each of the inflow and outflow tanks; the outflow tank further comprising an outflow valve positioned on an outflow side, above the outflow aperture; and a sample cylinder, having corresponding attachment components to secure to the attachment component on the outer face of both the inflow tank and the outflow tank and defining a water tight seal between said cylinder and said attachment component.

In a further preferred embodiment, the horizontal permeability device wherein the cylinder comprises an internal diameter of about the same diameter as the aperture and outflow aperture, wherein fluid filled into the inflow tank passes through the sample chamber and, upon sufficient volume of fluid, passes out of the outflow valve. In one embodiment, the apparatus further comprising a screen and a perforated sheet at each end of said cylinder. In one embodiment, the apparatus wherein said screen and said perforated sheet are compressed against the outer wall of said inflow and outflow tanks. In one embodiment, the apparatus wherein said screen and perforated sheet are stainless-steel. In one embodiment, the apparatus wherein said attachment components are an alignment block, said alignment block suitable for accepting an external surface of said cylinder, and wherein said corresponding attachment components are a gasket, said gasket is defined to fit between said alignment block and the external surface of said cylinder. In one embodiment, the apparatus wherein said inflow tank and outflow tank are made of a transparent plastic material. In one embodiment, the apparatus wherein said outflow valve can be opened or closed.

### BRIEF DESCRIPTION OF THE FIGURES

FIG. 1 depicts a schematic of an apparatus of the present invention.

FIGS. 2A and 2B wherein FIG. 2A depicts a detail of certain features of the apparatus. And FIG. 2B depicts a further detail of a sample cylinder and corresponding attachment components.

FIG. 3 depicts a sample preparation platform for use in the apparatus.

FIG. 4 depicts an apparatus for determining flow through a horizontal apparatus of the present disclosure, and depicting how volumes change over time as fluid flows through the apparatus.

FIG. 5 depicts a flow-chart of a method of calculating the permeability using the horizontal apparatus.

### DETAILED DESCRIPTION OF THE FIGURES

Soil permeability testing apparatus are used in a number of applications where a party is interested in evaluating soil permeability. For example, a non-disturbed core may be tested to evaluate the permeability at a predetermined location. In such case, special tools are utilized to take a core sample. In other cases, fill material is evaluated in view of its prospective use. The prior art details several different vertical chamber testing apparatus. However, in many applications these are simply non-functional for the tests that need to be performed. Indeed, frequent clogging of tubes leads to inconsistent or simply incorrect data.

Prior art devices typically use vertical chambers and narrow tubing to connect the soil sample to a water sample. However, on introduction of water or bacterial loads into these vertical devices, narrow tubing is often compromised, thus leading to uncertainty in the testing. By contrast, herein, is described a new permeability testing device using a horizontal testing chamber. Such a device can replace and be used in a plurality of situations wherein the vertical chamber was ineffective or impractical.

Soil permeability is necessary for testing for many construction projects requiring use of any type of fill materials or evaluation of existing soil materials. For example, in the design of a soil liner for a water retention pond or landfill, concerns over the long-term permeability of the soil can be studied. In these types of fill, there is an expectation of some bacterial flow in the fluids. The device described herein, will allow for testing of permeability using materials that incorporate bacteria within the fluid sample, for testing. The horizontal device will allow for testing of a soil sample to determine permeability, and potential changes in permeability, for example, if the sample is expected to remain at low or high permeability over time and with exposure to fluids with bacteria.

In certain instances, depending on the use of the fill material, clogging in the soil, by bacterial growth or other small particulates, may or may not be a good thing. Essentially, whether clogging of soil pores is good or bad is entirely dependent on the situation. For example, if creating a pond liner or levee, low permeability is optimal. However, if the fill is for a drain field of a septic system, you want to avoid clogging, to allow for flow of the fluids.

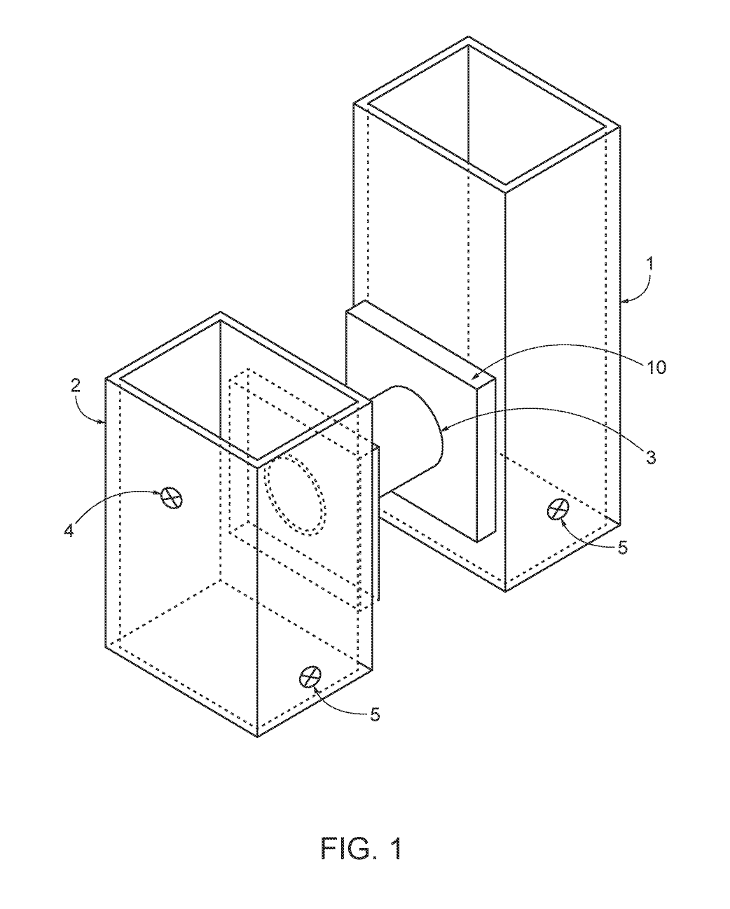

Accordingly, FIG. 1 depicts an overview schematic of a horizontal conductivity apparatus (soil permeability apparatus), having a large chamber (inflow chamber 1) on the right, and horizontal tube 3 (to hold the soil sample) connected thereto, and a smaller second chamber on the left (outflow chamber 2), as compared to the volume between the two chambers.

The equipment consists of two tanks (chambers) designed so that a sample of soil enclosed in a cylinder 3 can be placed between the inflow tank 1 and the outflow tank 2 and secured with the sample alignment block 10. Fluid can then be added to the tanks to saturate the sample, and fluid flow through the sample can be created by adding fluid to the inflow tank 1 so that the level of fluid in the inflow tank 1 is higher than the level of fluid in the outflow tank 2. The hydraulic conductivity can then be found by measuring the quantity of fluid that leaves the outflow tank 2, the time required for that quantity to leave the outflow tank 2 through the outlet valve 4, and the change in the differential height of the fluid in the inflow tank relative to the constant height of fluid in the outflow tank during that time. Fluids from either the inflow tank 1 or outflow tank 2 can be drained through a drain valve 5. A simple formula is utilized to calculate the conductivity.

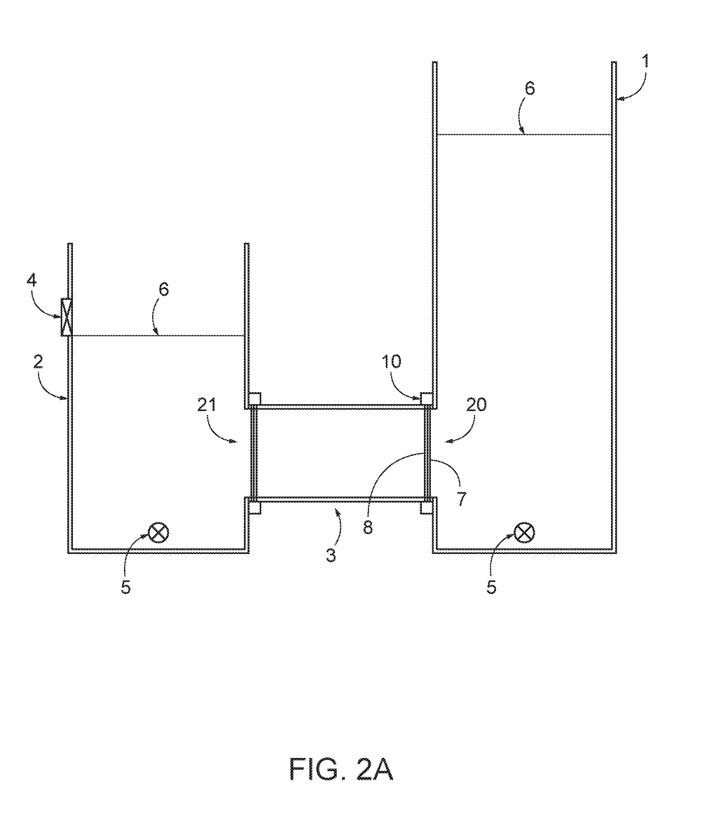

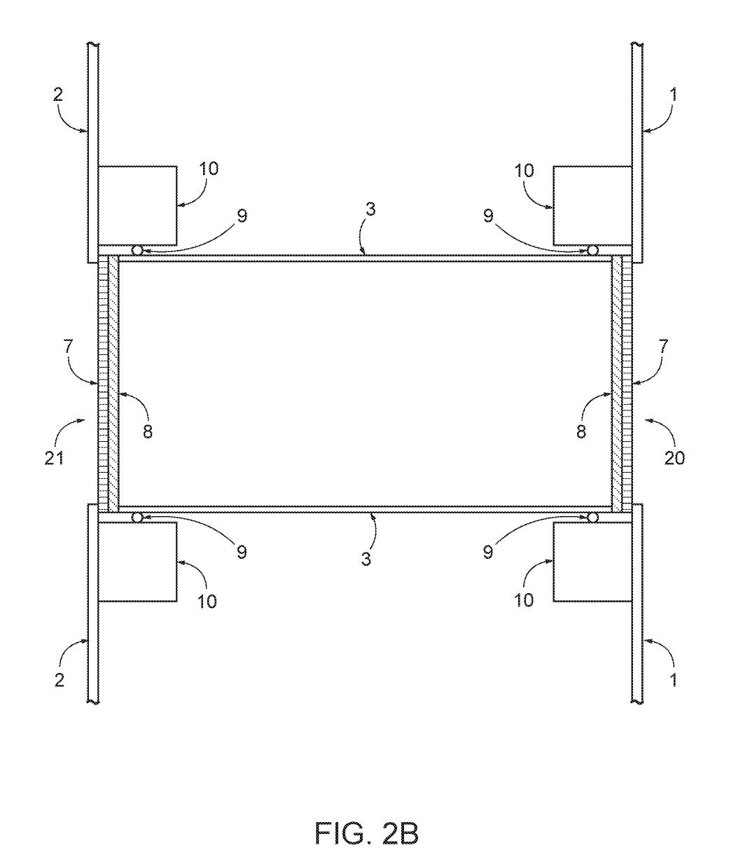

FIG. 2A provides a greater detail of an apparatus, comprising and inflow tank 1, an outflow tank 2, and a sample cylinder 3. The inflow and outflow tanks and cylinder may be made of any rigid material that can generate a water tight fit. For example, the tanks and cylinders may preferable be a plastic or acrylic material.

Within the inflow tank 1, there may include an optional a scale positioned vertically in the inflow tank 1 and allows for a visual determination of the change of fluid from a first point to a second point. For example the water line 6 is theoretically identified as a horizontal grey line within the inflow tank 1. This would be a hypothetical starting point for fluid within the inflow tank 1. And the water line 6 in the outflow tank 2, is the end point for fluid level. Fluid will flow via gravity from inflow tank 1 to outflow tank 2 and ultimately the water level in inflow tank 1 will stabilize with the water line 6 in outflow tank 2.

The inflow tank 1 has four sides creating a structure capable of retaining a fluid, and thus defines a height, a width, and a volume, with a closed base and an open top. Preferably this is a rectangular structure, though other shapes are suitable so long as volume is able to be calculated. The inflow tank 1 further comprises an aperture 20 on one side of the inflow tank 1 and the tank comprising connectivity means to secure a sample cylinder 3 around the aperture 20. The sample cylinder 3 is preferably a cylindrical vessel having an opening at each end, and is able to be secured to or adjacent to each of the inflow tank 1 and out flow tank 2 around the apertures 20 and 21. Indeed, as depicted in FIG. 2A, a stainless steel perforated sheet 7 and a stainless steel screen 8 are placed adjacent to one another at each end of the sample cylinder 3. The perforated sheet 7 and screen 8 are utilized to hold a sample material within said sample cylinder 3. In certain embodiments, the screen 8 is a metallic screen but it can be replaced with another filtering material suitable to maintain the particles in place, with said filtering material comprising natural or synthetic materials. In a preferred embodiment, the screen 8 is made of stainless-steel. Similarly, the perforated sheet is preferably a metallic material, but it can be made of another suitable material that is generally inert from the fluids being tested. In preferred embodiments, the metallic material is stainless steel. However, certain natural or synthetic materials, plastics, polymer, metals, metal alloys, or a combination of materials are suitable for either of the screen 8 or the perforated sheet 7.

In a preferred embodiment, when a sample is to be tested, the sample cylinder 3 is placed horizontally and one end of the sample cylinder 3 is attached over/around the aperture 20 in the inflow tank 1 while the opposing end is connected to the outflow aperture 21 in the outflow tank 2. The cylinder 3 simply covers the apertures 20 and 21 on each end to allow flow through the cylinder 3 between the two apertures 20 and 21. A gasket 9, as depicted in FIG. 2B, is utilized to secure the sample cylinder 3 to each of the inflow tank 1 and the outflow tanks 2 with a water tight seal between the external surface or external face of the sample cylinder 3 and the sample alignment block 10. Preferably, each of the inflow tank 1 and the outflow tank 2 contain sealing members (sample alignment blocks 10) that allow for securing the gasket 9 between said sealing members and the outer face of the sample cylinder 3. Additional sealing gaskets can be used by those of ordinary skill in the art, such as an O-Ring, or a rubber, polymeric, silicone, or plastic seal, or other gasket material or compression seal known to those of skill in the art, wherein the seal functions to secure the sample cylinder to the inflow 1 and outflow 2 tanks with a water tight seal under the pressures generated through the testing apparatus. If the system is pressurized for any reason, the seal will have to be able to handle the increased pressure as compared to the relatively lower pressures from the height of the water in gravity flow systems.

The outflow tank 2 contains an outflow valve 4 positioned above the opening in the outflow tank 2 that receives one end of the sample cylinder 3. This is done so that when the sample cylinder is positioned between the inflow 1 and outflow tanks 2, and filled with a sample material, the level of fluid in each of the inflow 1 and outflow tanks 2 is above the sample cylinder 3, thus ensuring that the sample remains saturated with fluid.

After a test is complete, fluid (typically water, but other fluids and contaminants may be included) will remain at just below the outflow valve 4, while maintaining saturation of the soil sample within the sample cylinder 3. If draining is needed, then the drain valves 5 can be opened on each of the inflow 1 and outflow tanks 2, to drain the liquid (fluid) within the inflow tank 1 and outflow tank 2. A new sample, or the same sample can be then placed into the testing apparatus and said liquids run through the apparatus.

FIG. 2B provides a further detail of the sample cylinder 3, as it attaches to the aperture 20 in the inflow tank 1 and the outflow aperture 21 in the outflow tank 2. Depicted is perforated sheet 7 and a screen 8, placed adjacent to one another to secure the soil sample in the horizontal sample cylinder 3. Depicted are O-rings 9, which contact the sample alignment block 10 to create a water tight seal and allow fluid flow through the aperture 20 into the outflow aperture 21 in the outflow tank 2. A portion of the perforated sheet 7 contacts with the outer wall of the inflow tank 1 and outflow tank 2, thereby securing these components in place. In other embodiments, these can be further secured with a threaded attachment or other known mechanism suitable for securing such components. The diameter of the apertures 20 and 21 can be the same as the inner diameter of the cylinder 3 in certain embodiments, though inner diameters of the cylinder 3 can be larger or smaller than the diameters of the apertures 20 and 21, and the gasket 9 is then utilized to fill some of the space to maintain a water tight fit.

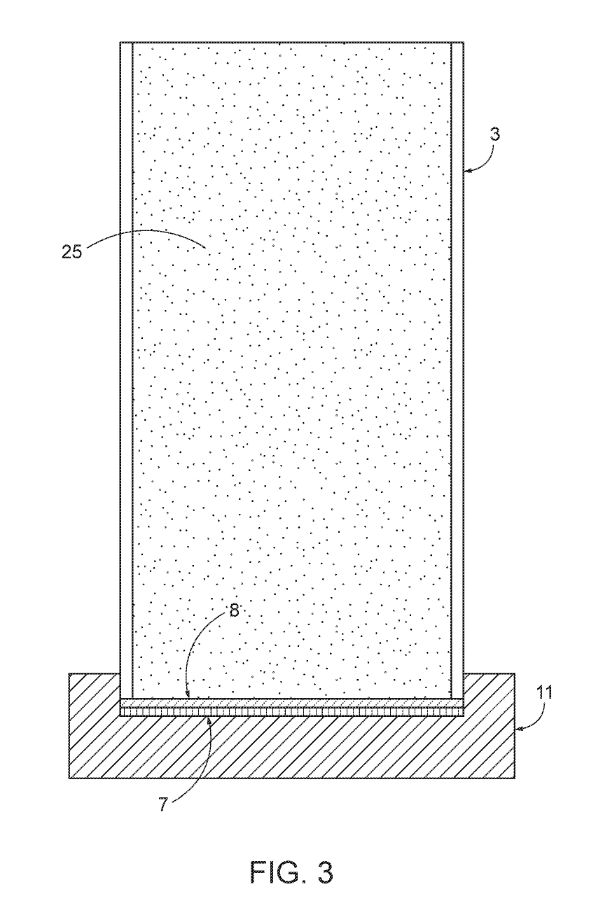

FIG. 3 depicts sample preparation, wherein a soil sample 25 (material within the sample cylinder 3) is placed within the sample cylinder. To aid in preparing the sample, a sample preparation platform 11 is utilized, which secures to the sample cylinder 3 and allows for a vertical orientation to load the sample. A perforated sheet 7, for example made from stainless-steel, is placed at the bottom, followed by the screen 8, for example also of stainless-steel, directly adjacent to and above the perforated sheet 7. The soil sample can then be added, wherein the soil will be held by the perforated sheet 7 and the screen 8. Once the sample cylinder 3 is filled to the amount of soil for a test to proceed, a screen 8 can be placed on the soil, and a perforated sheet 7 placed on top of the screen 8. Optionally, a gasket 9, like an O-ring, can be utilized to secure the sample cylinder 3 to the platform. After sample preparation, the properties of the soil sample would normally be measured (e.g., soil density) to determine whether the sample was representative of the conditions being modeled. If the desired properties were note achieved, the sample would be discarded, and another sample prepared.

The following provides an example of preparing a sample and running a test:

Creating the sample:

A sample is typically created in one of two ways. A sample is created in a first way wherein a reasonably undisturbed sample, is taken with a special tube sampling device. This is often used with samples that have a high clay content. The tube sampling device takes a core sample having a cylindrical shape, similar to the sample cylinder 3 as FIG. 2A. The cylindrical core sample typically includes a containing cylinder around the core. The horizontal device of the present disclosure is configured to allow for this containing cylinder to function as the sample cylinder 3, and therefore to be affixed between the inflow tank 1 and the outflow tank 2, using the gasket 9 or other attachment device. The perforated sheets 7 and screens 8 can be replaced with other suitable materials configured to the cylindrical core or added as necessary for use in the present horizontal device. Use of these exchangeable fittings, including the gasket 9, allows for direct attachment of this undisturbed sample for testing.

A sample is created in a second way through creation in the lab, or field, where the goal is to replicate the properties of the fill material. Accordingly, a sample of the fill material is placed into the sample cylinder 3, and then the fill material is compressed, such as being vibrated or pounded to get the same relative density as the fill material would have in the installation. Typically, to fill, the sample cylinder 3 is placed on the sample preparation platform (see FIG. 3), or otherwise compressed or prepared for horizontal permeability testing.

To allow the soil 25 to fill the cylinder the perforated sheet 7 and screen 8 are positioned at the bottom of the cylinder 3; and adding soil 25 to the cylinder 3 using whatever method is appropriate for the test being performed (e.g., adding moist soil in layers and compacting using tamping or adding dry soil in layers and compacting using vibration). Then leveling the top of the sample and placing the screen 8 and perforated sheet 7 on top of the sample.

The perforated sheet 7 provides a rigid end to the sample and the screen 8 prevents soil particles from moving out of the sample cylinder 3.

A sample can be created in other ways, including by capturing undisturbed samples. Devices suitable to capture such samples are known to those of ordinary skill in the art. Accordingly, a sample vessel can be configured to accept undisturbed samples from a site could be obtained using a Shelby tube sampler or other thin-walled sampling device. Accordingly, the apertures 20 and 21 between the inflow tank 1 and the outflow tank 2 and the sheets 7 and screens 8 can be sized to accept such sampling tubes in a standard size. Alternatively, different sized gaskets 9 or can be utilized to accept sample cylinders 3 of varying diameters without having to modify the size of the apertures 20 or 21 or of the alignment blocks 10.

Placing sample in test apparatus:
a. Lift cylinder with soil sample off the sample preparation platform;
b. Place cylinder with soil sample over the aperture 20 on the inflow tank 1 (the inflow tank 1 can be either in a normally vertical position, or in a horizontal position with the aperture 20 for the sample facing upwards). This forces the screen 8 and perforated sheet 7 against the outer face of the inflow tank and thus secures the screen 8 and perforated sheet 7 into place;
c. Place the outflow tank 2 to accept the sample cylinder 3 over the outflow aperture 21, whether this is in a vertical or in a horizontal position and place it on the cylinder with the soil sample. This forces the screen 8 and perforated sheet 7 against the outer face of the outflow tank and thus secures the screen 8 and perforated sheet 7 into place;
d. Where the inflow tank 1 and outflow tank 2 are horizontal, simultaneously, move inflow tank 1, cylinder 3, and outflow tank 2 so that the tanks are in a vertical position and the cylinder is horizontal (see FIG. 1).

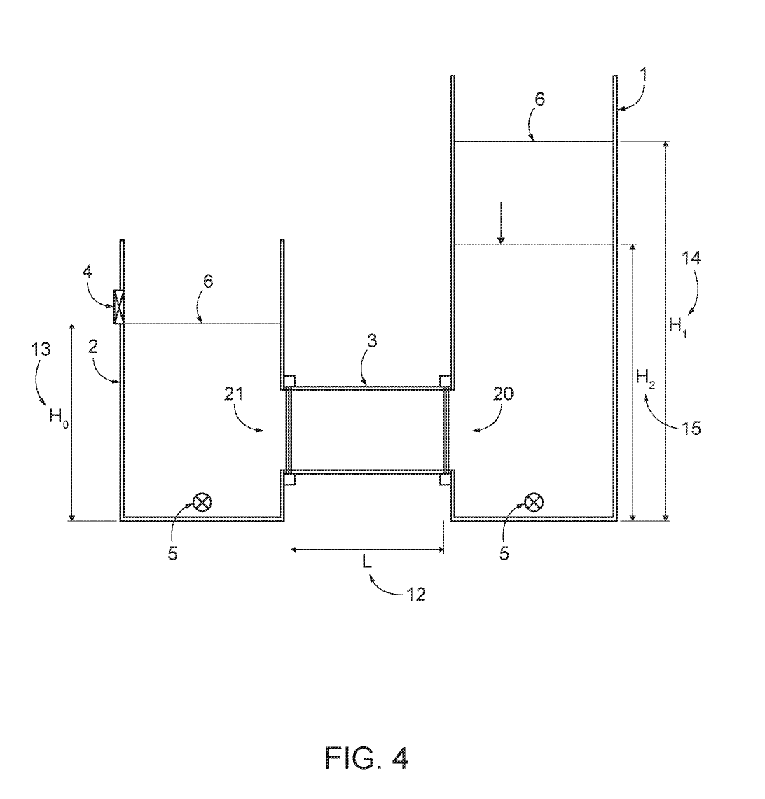

Running the test, for example as depicted FIG. 4:
a. With the drain valves 5 closed, fluid is added to the inflow tank 1 and allowed to flow through the aperture 20 into the cylinder 3 and through the sample and exiting the outflow aperture 21 and into the outflow tank 2. Fluid is added until the levels in the two tanks equalize due to the presence of and at the height of the outflow valve 4. The outflow valve 4 remains open and establishes the constant level of fluid in the outflow tank 2. The level of the fluid in the outflow tank is H₀ 13. This condition is maintained until the soil is fully permeated with the fluid.

b. Two heights are identified on the inflow tank, H1 14 and H2 15 (H1 15>H2 15)>H0 13.

c. Fluid is then added to the inflow tank 13 so that the initial level of fluid is higher than H1, e.g. a fluid line 6.

d. Because of the differential levels of fluid between the inflow tank 1 and the outflow tank 2, flow will occur through the sample and the level of the fluid in the inflow tank 1 will begin to decrease, as fluid exits the outflow valve 4 in the outflow tank 2.

e. When the level of the fluid in the inflow tank 1 reaches H1 14, a stopwatch is started and the fluid leaving the outflow tank 2 is captured in a graduated cylinder. If the volume of fluid between H1 14 and H2 15 in the inflow tank is known, it is not necessary to collect the fluid leaving the outflow tank 2.

f. When the level of the fluid in the inflow tank 1 reaches H2 15, the time (t) is recorded from the stopwatch and the total volume of fluid captured in the graduated cylinder (Q) is recorded.

FIG. 4 further defines the data obtained during the test via a formula as follows:

L 12=Length of soil sample

A=Cross-sectional area of soil sample, Area=3.1416*(internal radius of cylinder 3)²

Q=Volume of fluid that moves through sample during test

t=length of time of test

H1 14—Height of the fluid sample at start of test

H2 15—Height of the fluid sample at end of test

H0 13—Height of the fluid sample at stasis.

$$k = \frac{Q L}{A t ( {H}_{1} - {H}_{2} )} ln [ \frac{( {H}_{1} - {H}_{0} )}{( {H}_{2} - {H}_{0} )} ]$$

These tests allow for collection of data from any number of fluids passed through the sample. Furthermore, because of the design of the horizontal sample cylinder 3, the sample can remain saturated with the sample fluid, and tests run and re-run any number of times, over an undefined period of time. Indeed, samples can be run once a day, twice a day, or as many times a day as necessary, including constant running of sample fluids (e.g. through use of a pump to recirculate fluids from outflow tank 2 back to inflow tank 1, or simply through fluid being constantly added to inflow tank 1). Alternatively, samples can be run once a week, once a month, or as frequently, or infrequently as determined by the protocol for soil testing. A primary goal of the permeability devices is to figure out how a fluid will impact the soil sample over time. So we set up the fluid, so both tanks are full and that the soil is saturated—then we provide nutrients on a regular basis, to model leachate field of infusion, then when we feed the fluids, we raise the upstream side, we watch the time to go between two levels, so we can see if this changes over time.

So, we can have steady state of no flow for a long period. Steady state of constant flow, or intermittent flow—The soil sample you choose, the length of time you test, the fluid you choose, are chosen to replicate the field conditions, or what you expect them to be during the project.

The horizontal flow testing device as described herein provides a significant advantage over the prior art, as in instances where the soil is tested over a long period of time, bacterial growth is inevitable. In the prior art systems, clogs in the tubing system are frequently generated and cannot be resolved without disturbing the soil sample. Any disruption of the soil sample invalidates the data, as the results may be due to soil disturbances and not the soil properties. Similarly, clogs in the pipes impact the outcome of the test so you can't tell whether the permeability has changed, or the system is clogged.

In the horizontal system of the present disclosure, the tanks can be cleaned and thus keep normalized flow of fluids so we eliminate the use of pipes and therefore the chance of clogs in pipes leading to changes in our data.

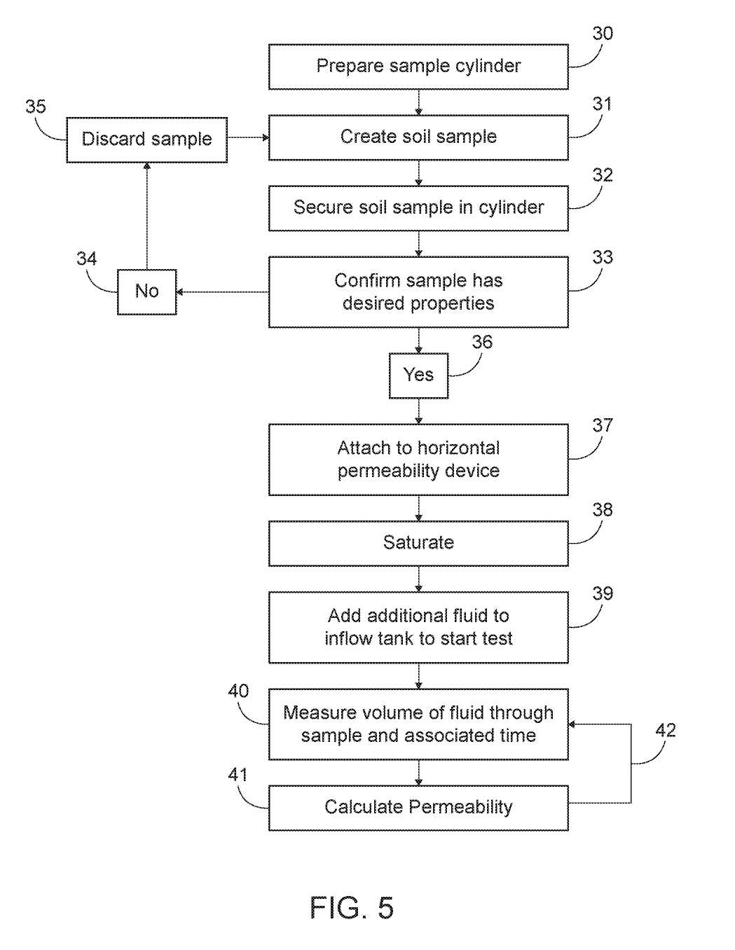

FIG. 5 details a flow-chart of sample testing. A sample cylinder 3 is prepared as indicated above 30 or through a commercial means. If soil 25 needs to be filled, the sample is filled 31. The soil sample 25 is secured within the sample cylinder 32. The sample is then examined to ensure the desired properties were achieved 33. If the sample does not have the desired properties 34, then the sample is discarded 35 and a new sample created 31.

If the sample has the desired properties 36, the sample cylinder 3 is secured 37 to the horizontal permeability device, between the inflow chamber 1 and the outflow chamber 2. Fluid is added 38 to saturate the soil sample.

After saturation, then testing fluid 39 is added to the inflow tank 1 and the test can begin. Fluid can be captured, and time measured 40 to calculate permeability 41. Ultimately, the process can be repeated 42 by adding testing fluid to the inflow tank 1.

### EXAMPLE 1

### Sample of Data Obtained

A test was conducted on prototype equipment on Jun. 23, 2017. The following data were observed:

A=cross-sectional area of soil sample=45.36 cm2

L=length of sample=18.18 cm

H₀=21.9 cm; H₁=45 cm; H₂=42.3 cm Q=400 cm3

T=84.3 sec

Calculation of permeability: FIG. 4 shows the equation to calculate permeability. For the data obtained during the sample test, the permeability is calculated as:

$$k = \frac{Q L}{A t ( {H}_{1} - {H}_{2} )} ln [ \frac{( {H}_{1} - {H}_{0} )}{( {H}_{2} - {H}_{0} )} ]$$

$$k = \frac{( 400 {cm}^{3} ) ( 18.18 cm )}{( 45.36 {cm}^{2} ) ( 84.3 sec )} ln [ \frac{( 45 cm - 21.9 cm )}{( 42.3 cm - 21.9 cm )} ] = 0.0876 cm / sec$$

Having now described the horizontal permeability testing device, it is evident that modifications can be made, by those of ordinary skill in the art, without deviating from the scope of the invention. Indeed, the particular materials and attachment means between the components can be modified based on the size of the components, the weight of a sample, and the like. Furthermore, it is suitable to attach a drain line or water access line to one or both chambers to allow for constant flow testing of a soil sample.

What is claimed is:

1. An apparatus for testing permeability of a soil sample comprising: an inflow chamber, an outflow chamber, each chamber having a closed base, a side, and an open top, and a sample cylinder, said inflow chamber comprising an aperture disposed on said side of the inflow chamber, and the outflow chamber having a corresponding outflow aperture on said side of the outflow chamber; each of the inflow chamber and outflow chamber comprising attachment means disposed of on an outer face of the side, adjacent to said aperture and outflow aperture, to accept and secure thereto said sample cylinder, wherein said sample cylinder is positioned to allow passage of fluids through the aperture and outflow aperture between the inflow chamber and outflow chamber.

2. The apparatus of claim 1, said inflow chamber further comprising a drain valve positioned below the aperture disposed of on said inflow chamber, and said outflow chamber having both a drain valve positioned below the outflow aperture and an outflow valve positioned above the outflow aperture in the outflow chamber.

3. The apparatus of claim 1, wherein said sample cylinder is open at each end of the cylinder, and is oriented in a horizontal manner between said inflow chamber and said outflow chamber.

4. The apparatus of claim 3, wherein said open at each end of the cylinder comprises a perforated sheet and a screen disposed of adjacent to one another.

5. The apparatus of claim 4, wherein the screen is positioned inside of the perforated sheet, and wherein the perforated sheet engages against the outer face on the side of the inflow chamber and the outflow chamber to maintain the perforated sheet and screen in position.

6. The apparatus of claim 5, wherein the attachment means is an alignment block defining a circumferential perimeter on the outer face of the side and having a diameter larger than the aperture and outflow aperture, and a gasket being disposed of between the alignment block and the sample cylinder, which maintains the sample cylinder between the inflow tank and the outflow tank.

7. The apparatus of claim 6 wherein a soil sample is positioned within the sample cylinder.

8. The apparatus of claim 6, wherein the gasket is an O-ring.

9. The apparatus of claim 5, wherein the perforated sheet and the screen are made of stainless-steel.

10. A method of determining permeability of a sample of soil comprising:
a. placing a perforated sheet into a sample cylinder, said sample cylinder having a first and second opening at each end of said cylinder, and placing a screen into the cylinder, wherein said perforated sheet and screen are adjacent to one another at one end of said sample cylinder;
b. filling a sample of soil into a sample cylinder;
c. compacting the soil within said sample;
d. placing a second screen on top of said compacted soil, and placing a second perforated sheet adjacent to said second screen;
e. positioning said sample cylinder between an inflow chamber and an outflow chamber, said inflow chamber and outflow chamber comprising an aperture on said inflow chamber and an outflow aperture on said outflow chamber, said cylinder positioned wherein the first and second openings are engaged to allow passage of fluids through the cylinder between the aperture and outflow aperture;
f. securing said sample cylinder between said inflow chamber and outflow chamber to be water tight;
g. filling said inflow chamber with a fluid to be tested sufficient to fill the inflow tank and outflow tank to a level to saturate the soil sample;
h. adding additional fluid to said inflow chamber and measure the rate of decrease of the water level in said inflow chamber; and
i. calculating the permeability of said soil sample using the rate of decrease of the water level.

11. The method of claim 10, wherein the permeability of said soil is calculated by:
$$k = \frac{Q L}{A t ( {H}_{1} - {H}_{2} )} ln [ \frac{( {H}_{1} - {H}_{0} )}{( {H}_{2} - {H}_{0} )} ] ,$$
wherein L=Length of soil sample, A=Cross-sectional area of soil sample, Q=Volume of fluid that moves through sample during test, t=length of time of test, H1=Height of the fluid sample at start of test, H2=Height of the fluid sample at end of test, and H0=Height of the fluid sample at stasis.

12. The method of claim 10, wherein said fluid comprises bacteria.

13. A horizontal permeability apparatus comprising an inflow tank and an outflow tank, each having a closed base, four sides, each side having an inner and outer face, an open top, and a height, and an outflow tank, comprising an outflow base, four outflow sides, each side having an inner and outer face, an open top and having a height less than the inflow tank; each inflow and outflow tank comprising an aperture disposed on a side on the inflow tank and an outflow aperture on the outflow tank, said aperture and outflow aperture having attachment components disposed of adjacent to and around said aperture on the outer face of each of the inflow and outflow tanks; the outflow tank further comprising an outflow valve positioned on an outflow side, above the outflow aperture; and a sample cylinder, having corresponding attachment components to secure to the attachment component on the outer face of both the inflow tank and the outflow tank and defining a water tight seal between said cylinder and said attachment component.

14. The horizontal permeability device of claim 13 wherein the cylinder comprises an internal diameter of about the same diameter as the aperture and outflow aperture, wherein fluid filled into the inflow tank passes through the sample chamber and, upon sufficient volume of fluid, passes out of the outflow valve.

15. The horizontal permeability device of claim 13 further comprising a screen and a perforated sheet at each end of said cylinder.

16. The horizontal permeability device of claim 15, wherein said screen and said perforated sheet are compressed against the outer wall of said inflow and outflow tanks.

17. The horizontal permeability device of claim 15, wherein said screen and perforated sheet are stainless-steel.

18. The horizontal permeability device of claim 13, wherein said attachment components are an alignment block, said alignment block suitable for accepting an external surface of said cylinder, and wherein said corresponding attachment components are a gasket, said gasket is defined to fit between said alignment block and the external surface of said cylinder.

19. The horizontal permeability device of claim 13, wherein said inflow tank and outflow tank are made of a transparent plastic material.

20. The horizontal permeability device of claim 13, wherein said outflow valve can be opened or closed.
